(19) United States
(12) Patent Application Publication
(10) Pub. No.: US 2003/0027209 A1
(43) Pub. Date: Feb. 6, 2003

(54) Methods for producing and improving therapeutic potency of binding polypeptides

(76) Inventor: William D. Huse, Del Mar, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/001,202

(22) Filed: Oct. 30, 2001

Related U.S. Application Data
(60) Provisional application No. 60/367,372, filed on Oct. 30, 2000.

(51) Int. Cl. G01N 33/53; A61K 39/395; C07K 16/44

(52) U.S. Cl. 435/7.1; 530/388.15; 424/141.1

(57) ABSTRACT

The invention provides a binding polypeptide, or functional fragment thereof, comprising a kₒₙ of at least about 9×10⁷ M⁻¹s⁻¹ for associating with a ligand and having therapeutic potency. The invention also provides a method of determining the therapeutic potency of a binding polypeptide. The methods consist of (a) contacting a binding polypeptide with a ligand; (b) measuring association rate for binding between the binding polypeptide and the ligand, and (c) comparing the association rate for the binding polypeptide to an association rate for a therapeutic control, the relative association rate for the binding polypeptide compared to the association rate for the therapeutic control indicating that the binding polypeptide will exhibit a difference in therapeutic potency correlative with the difference between the association rates.

This application is based on, and claims the benefit of, U.S. Provisional Application No. 60/ ______, filed Oct. 30, 2000, which was converted from U.S. Ser. No. 09/702,140, and which is incorporated herein by reference.

### BACKGROUND OF THE INVENTION

The present invention relates generally to the treatment of disease and more specifically to binding molecules useful as therapeutics.

Modern medicine benefits from increased manipulation of molecular level interactions that mediate individual diseases. This is especially the case for treatment of disease and disease symptoms with drug therapies. The drug development industry uses strategies based on molecular level analysis in attempting to develop therapeutically effective drugs.

One such strategy, used in the drug development industry, is to identify a target molecule associated with a disease and to produce a drug that binds to the target molecule to either block the target molecule's activity or to deliver a toxic payload to the site where the target molecule resides in the diseased individual. Under such a strategy, the discovery phase of research utilizes in vitro methods to identify a lead drug candidate that binds to a target molecule. The lead drug candidate can then be entered into the validation phase of research where in vivo tests are performed to determine if the lead drug candidate demonstrates therapeutic effectiveness.

Two commonly used discovery phase approaches are structure based drug design and screening a pool of candidate molecules. Structure based drug design uses the target molecule's three dimensional structure, or other structure-related property, as a template to which drug candidates are fit to identify a structural model for a lead drug candidate. The lead drug candidate is then synthesized and tested in vitro. Alternatively, screening uses an isolated target molecule to select a lead drug candidate from a large population of drug candidates in vitro. One factor in both approaches is exploitation of the stability of the binding interaction between the target molecule and lead drug candidate. In this regard a large number of structure based design algorithms are aimed at identifying a lead drug candidate that docks with the target molecule to form a stable complex and a large number of screens are designed to select lead drug candidates that form a stable binding complex with the target molecule.

Genomics, protein engineering and combinatorial chemistry have been used to identify targets and potential drug candidates that are input into the in vitro methods of discovery phase research. These and other methods may allow high throughput identification and production of therapeutic drugs leading to increases in both the number of disease targets and the number of lead drug candidates.

Unfortunately, the production of therapeutic drugs has not improved in a correlative fashion with improvements in methods of discovery phase approaches or the greater number and variety of discovery phase inputs. In particular, the identified lead drug candidates too often fail to demonstrate therapeutic effectiveness. Diversion of resources to an unsuccessful drug candidate in the validation phase can be costly because millions of dollars and numerous years can be wasted on a failed lead drug candidate. More importantly, those suffering from devastating diseases are deprived of a treatment or cure.

Thus, there exists a need for a rapid and efficient method which accurately predicts successful lead drug candidates exhibiting therapeutic effectiveness against a disease. The present invention satisfies this need and provides related advantages as well.

### SUMMARY OF THE INVENTION

The invention provides a binding polypeptide, or functional fragment thereof, comprising a kₒₙ of at least about 9×10⁷ M⁻¹s⁻¹ for associating with a ligand and having therapeutic potency. The invention also provides a method of determining the therapeutic potency of a binding polypeptide. The methods consist of (a) contacting a binding polypeptide with a ligand; (b) measuring association rate for binding between the binding polypeptide and the ligand, and (c) comparing the association rate for the binding polypeptide to an association rate for a therapeutic control, the relative association rate for the binding polypeptide compared to the association rate for the therapeutic control indicating that the binding polypeptide will exhibit a difference in therapeutic potency correlative with the difference between the association rates.

### DETAILED DESCRIPTION OF THE INVENTION

The invention is directed to the discovery that the therapeutic potency of a molecule correlates with the rate at which the therapeutic molecule associates with a ligand that mediates or correlates with a pathological condition. The invention provides a binding polypeptide, or functional fragment thereof, having a kₒₙ of at least about 9×10⁷ M⁻¹s⁻¹ for associating with a ligand and having therapeutic potency. The invention further provides a grafted antibody, or functional fragment thereof, having a kₒₙ of at least about 1.3×10⁶ M⁻¹s⁻¹ to a ligand and having therapeutic potency.

In one embodiment the methods of the invention allow for accurate in vitro prediction and identification of molecules having therapeutic potency. In one embodiment the methods involve determining the therapeutic potency of a binding polypeptide by comparing the association rate for the binding polypeptide to an association rate for a therapeutic control. The binding polypeptide will exhibit a difference in therapeutic potency correlative with the difference between said association rates. In another embodiment, the methods of the invention involve identifying therapeutic potency of a binding polypeptide by identifying a binding polypeptide exhibiting a high association rate or kₒₙ, correlating with its therapeutic potency. The methods of the invention can also be used to change the structure and ligand binding activity of a parent polypeptide to create one or more progeny polypeptides, and to identify progeny polypeptides that are binding polypeptides having improved therapeutic potency resulting from increased association rate with a ligand.

An advantage of the invention is that a binding polypeptide having improved therapeutic potency can be distinguished from a binding polypeptide that has an increased Kₐ for a ligand but not improved therapeutic potency. A further advantage of the methods of the invention is that a means to screen large numbers of potential therapeutic molecules in vitro is provided, thereby increasing the rate and efficiency of identifying effective therapeutics while reducing the costs associated with in vivo testing of failed therapeutics.

As used herein, the term “binding polypeptide” refers to a polymer of amino acids that selectively associates with a ligand. A binding polypeptide can have, for example, at least 2, 5, 8, 10, 12, 15, 20, 25, 50, 100, 200 or 400 or more amino acids so long as the polypeptide retains ability to associate with a ligand. Therefore, the term binding polypeptide, as used herein, includes all sizes of amino acid polymers ranging from a couple to hundreds or even thousands of amino acids.

A binding polypeptide can be a naturally occurring polypeptide, for example, a receptor, enzyme or hormone. A receptor can include, for example, an immunoglobulin, such as an antibody or T cell receptor; integrin; hormone receptor; lectin; membrane receptor; or transmitter receptor. An enzyme can include, for example, a protease, oxidoreductase, kinase, lipase, phosphatase, DNA modifying enzyme, polymerase, caspase, transcription factor, GTPase, ATPase, or a membrane channel. A hormone can include for example, a growth factor, insulin, cytokine, neural peptide, extracellular matrix protein or clotting factor. A binding polypeptide can be a modified form of a naturally occurring polypeptide, for example, a fragment, chimera containing amino acids from a donor polypeptide, or fusion of fragments from one or more donor polypeptides so long as such polypeptide retains ability to associate with a ligand.

A binding polypeptide can be a polypeptide that contains a non-naturally occurring moiety including, for example, an amino acid derivative, sterioisomer of an amino acid, amino acid analogue or functional mimetic of an amino acid. For example, a derivatives can include a chemical modification of the polypeptide such as alkylation, acylation, carbamylation, iodination, or any modification which derivatizes the polypeptide. An analogue can include a modified amino acid, for example, hydroxyproline or carboxyglutamate, and can include an amino acid that is not linked by a peptide bond. Mimetics encompass a molecule containing a chemical moiety that mimics the function of the polypeptide regardless of a difference in three-dimensional structure between the binding polypeptide and mimetic. For example, if a polypeptide contains two charged chemical moieties in a functional domain, a mimetic can place two charged chemical moieties in a spatial orientation and constrained structure so that the relative location of the charged chemical moieties is maintained in three-dimensional space independent of any other differences between the polypeptide and mimetic.

As used herein, the term “ligand” refers to a small molecule, compound or macromolecule that can selectively associate with a binding polypeptide. A ligand can be a naturally occurring molecule, compound or macromolecule including, for example, DNA, RNA, polypeptide, lipid, carbohydrate, amino acid, nucleotide or hormone. A ligand can be a derivative of a naturally occurring molecule, compound or macromolecule resulting in, for example, an added moiety, a removed moiety or a rearrangement in the relative location of moieties. Examples of added moieties include, for example, a biotin, peptide such as polyhistidine, radioisotope or chemically reactive group capable of forming a covalent bond to a second molecule. A ligand can be a mimetic of naturally occurring molecule, compound or macromolecule. Mimetics encompass molecules containing chemical moieties that mimic the function of the ligand regardless of differences between three-dimensional structure of the mimetic and the ligand. A mimetic can be, for example, a synthetically prepared molecule or a polypeptide containing a modified form of a naturally occurring amino acid. A ligand can be an antigen found on a cell such as a cancer cell, microbe, bacteria, fungus or virus. A ligand can also be a molecule that is a toxic substance.

As used herein, the term “parent polypeptide” refers to a polymer of amino acids that can be changed to produce a binding polypeptide. Therefore, a parent polypeptide is the molecule to be improved using the methods of the invention. As used herein a parent polypeptide can have, for example, at least 2, 5, 8, 10, 12, 15, 20, 25, 50, 100, 200 or 400 or more amino acids. Therefore, the term parent polypeptide, as used herein, includes all sizes of amino acid polymers ranging from a couple to hundreds or even thousands of amino acids.

A parent polypeptide can be a naturally occurring polypeptide, for example, a receptor, enzyme or hormone such as those described above in reference to a binding polypeptide. A parent polypeptide can be a polypeptide that contains a non-naturally occurring moiety including, for example, an amino acid derivative, a sterioisomers of an amino acid, an amino acid analogue or a functional mimetic of an amino acid such as those described above in reference to a binding polypeptide.

As used herein the term “progeny polypeptide” refers to a polymer of amino acids that has different structure compared to the parent polypeptide from which it was produced. A different structure can include, for example, addition, deletion, substitution or chemical modification of one or more amino acids. A progeny polypeptide can be a different species from the parent polypeptide. A progeny polypeptide can associate with a ligand at the same or different association rate compared to the association rate at which its parent polypeptide associates with the same ligand. As used herein a progeny polypeptide can have, for example, at least 2, 5, 8, 10, 12, 15, 20, 25, 50, 100, 200 or 400 or more amino acids. Therefore, the term progeny polypeptide, as used herein, includes all sizes of amino acid polymers ranging from a couple to hundreds or even thousands of amino acids.

A progeny polypeptide can be a modified form of a parent polypeptide, for example, a fragment, chimera containing amino acids from a donor polypeptide, or fusion of fragments from one or more donor polypeptide. A progeny polypeptide can be a polypeptide that contains a non-naturally occurring moiety including, for example, an amino acid derivative, sterioisomer of an amino acid, amino acid analogue or functional mimetic of an amino acid such as those described above.

As used herein, the term “grafted” when used in reference to an antibody, or functional fragment thereof, refers to an antibody, or functional fragment thereof, having a variable region acceptor framework from one species containing one or more CDR from a donor or second species. One skilled in the art will know that the function of an antibody, or functional fragment thereof, can be influenced by a change in a single CDR or more preferably in multiple CDRs. Amino acids can be added, deleted or substituted at any position in the acceptor framework or donor CDRs and can include, for example, changes that modify structure or function of the grafted antibody, or functional fragment thereof, whether minor or significant so long as the antibody, or functional fragment thereof, contains a variable region acceptor framework from one species and at least one CDR from another species. Description of grafted antibodies and methods for their production are well known in the art and are described, for example, in U.S. Pat. No. 5,225,539; “Protein Engineering of Antibody Molecules for Prophylactic and Therapeutic Applications in Man,” Clark, M. (ed.), Nottingham, England: Academic Titles (1993); Winter and Harris, Immunol. Today, 14:243-246 (1993); Winter and Harris, Tips, 14:139-143 (1993) and Couto et al. Cancer Res., 55:1717-1722 (1995) which are incorporated herein by reference.

As used herein, the term “functional fragment,” when used in reference to a binding polypeptide, is intended to refer to a portion of a binding polypeptide which retains the ability to selectively associate with a ligand. Functional fragments can include dissociated subunits of a binding polypeptide, for example, individual heavy or light chains of an antibody. Functional fragments can include portions of a binding polypeptide having a reduced number of amino acids, for example, Fd, Fab or F(ab)₂ portions of an antibody. Functional fragments can include portions of a dissociated subunits of a binding polypeptide having a reduced number of amino acids including, for example, Fv, V_{H}, a CDR, or scFv portions of an antibody. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990), which are incorporated herein by reference. Thus, a functional fragment can include an immunologically active portion, fragment or segment of an antibody.

A functional fragment of a binding polypeptide can have minor structural differences in comparison to a full length binding polypeptide so long as the fragment has about the same structure as the corresponding region of the full length binding polypeptide and retains the ability to selectively associate with a ligand. Minor structural differences can be at the primary, secondary, tertiary, or quaternary sequence level. Structural differences at the primary sequence level include changes in the amino acid sequence and can be, for example, additions, deletions or substitutions of amino acids or chemical modifications of amino acids, such as addition of a chemical moiety, so long as such a polypeptide retains the ability to associate with a ligand. An added moiety can include, for example, a chemically derivatized amino acid, D-stereoisomer of an amino acid, non-naturally naturally occurring amino acid, amino acid analogue or a mimetic of an amino acid. Structural differences between a binding polypeptide and functional fragment thereof at the secondary level including, for example, a change in alpha helix, loop or beta sheet structure can occur so long as the resulting functional fragment retains the ability to associate with a ligand. A functional fragment of a binding polypeptide can also have a structural difference at the tertiary level including, for example, a change in the relative location of a secondary structure element or change in overall fold of the binding polypeptide. Structural differences at the quaternary level can include, for example, a change in the number of subunits in a binding polypeptide or a change in the interfaces at which subunits in a binding polypeptide interact so long as the functional fragment retains the ability to associate with a ligand.

As used herein, the term “complimentarity determining region” or “CDR” is intended to mean a non-contiguous antigen combining site found within the variable region of either a heavy or light chain polypeptides of an immunoglobulin. The term CDR region is well known in the art and has been defined by Kabat et al., U.S. Dept. of Health and Human Services, “Sequences of Proteins of Immunological Interest” (1983) and by Chothia et al., J. Mol. Biol. 196:901-917 (1987) and additionally by MacCallum et al., J. Mol. Biol. 262:732-745 (1996), which are incorporated herein by reference, and include overlapping or subsets of amino acid residues when compared against each other. Application of any of the above three definitions to refer to a CDR of an antibody, or functional fragment thereof, is intended to be within the scope of the term as defined and used herein. The appropriate amino acid residues which encompass the CDRs, as defined by each of the above cited references, are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 52-56 | 47-58 |
| V_{H} CDR3 | 95-102 | 95-102 | 93-101 |
| V_{L} CDR1 | 24-34 | 24-34 | 30-36 |
| V_{L} CDR2 | 50-56 | 50-56 | 46-55 |
| V_{L} CDR3 | 89-97 | 89-97 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “association rate” refers to the time in which binding polypeptide and ligand become bound to form a complex. Use of the term herein is intended to be consistent with the meaning of the term as it is known in the art. The association rate can be correlated with the time dependent appearance of a species composed of binding polypeptide bound to ligand, the time dependent disappearance of free binding polypeptide or the time dependent disappearance of free ligand in a mixture of species including binding polypeptide and ligand. Free binding polypeptide species refers to a binding polypeptide that is competent to bind at least one ligand and free ligand refers to a ligand that is competent to bind at least one binding polypeptide. The scope of the term association rate is intended to include kₒₙ. The association rate is known in the art to be proportional to kₒₙ and proportional to the product of Kₐ and k_{off}.

As used herein, the term “associating,” when used in reference to a binding polypeptide and ligand, is intended to refer to the process by which a binding polypeptide and ligand contact each other in a manner that results in the species of binding polypeptide bound to ligand. Use of the term associating is intended to be consistent with the meaning of the term as it is known in the art. The process is different from and can be distinguished by those skilled in the art from the reverse process by which the complex of binding polypeptide bound to ligand dissociates to yield free binding polypeptide and free ligand.

As used herein, the term “kₒₙ” refers to the association rate constant equating the association rate with the concentration of the free binding polypeptide and free ligand. The term kₒₙ is intended to be consistent with the meaning of the term as it is known in the art. Therefore, kₒₙ is a quantitative measure of association rate. For example, when binding polypeptide A and ligand B associate to form the bound species AB, the association rate will equal the kₒₙ multiplied by the product of the concentration of free binding polypeptide A multiplied by the concentration of free ligand B. A mathematical equation describing this relationship is: association rate=kₒₙ*[A]*[B] where [A] is the concentration of polypeptide A and [B] is the concentration of ligand B.

As used herein, the term “Kₐ” refers to the association constant and is intended to be consistent with the meaning of the term as it is understood in the art. The Kₐ is a measure of the strength, affinity and tightness of binding. Specifically Kₐ is an equilibrium constant equating the concentrations of free binding polypeptide, free ligand and binding polypeptide bound to ligand occurring at equilibrium. The Kₐ can be used to compare the affinity of different binding polypeptides for various ligands at equilibrium. For example, a binding polypeptide with a higher numerical value of Kₐ for binding a ligand compared to the Kₐ for a second binding polypeptide binding the same ligand is understood in the art to have higher affinity for that ligand. The Kₐ relates the association rate constant (kₒₙ) and the dissociation rate constant (k_{off}) according to the relationship Kₐ=kₒₙ/k_{off}. The k_{off} is the mathematical constant used in the art to quantitate the time for an associated binding polypeptide and ligand to separate. Accordingly, kₒₙ is the product of Kₐ and k_{off}. The mathematical inverse of Kₐ is known in the art as the K_{d} or dissociation constant. Therefore, K_{d}=1/Kₐ=k_{off}/kₒₙ. Thus, a binding polypeptide with a lower numerical value of K_{d} for binding a ligand compared to the K_{d} for a second binding polypeptide binding the same ligand is understood in the art to have higher affinity for that ligand.

As used herein, the term “therapeutic potency” is intended to refer to a predictive measure of efficacy or relative efficacy. If a binding polypeptide has therapeutic potency it produces a desired therapeutic effect. Therapeutic potency includes a kinetic property and is proportional to the association rate for a binding polypeptide associating with a ligand. As such, the term reflects the effect of expeditious association between a binding polypeptide and ligand that cures, alleviates, removes or lessens the symptoms of, or prevents or reduces the possibility of contracting a pathological condition. A binding polypeptide having an increased kₒₙ when associating with a ligand will display more expeditious association with a ligand thereby having improved therapeutic potency compared to a parent polypeptide or other polypeptide having a lower kₒₙ when associating with the same ligand.

As used herein, the term “therapeutic control” refers to a molecule to which a binding polypeptide can be compared when determining or identifying therapeutic potency and which is related to a pathological condition to which the binding polypeptide is targeted. A molecule can be related to a pathological condition, for example, by having demonstrated efficacy in treating the pathology, having demonstrated interaction with a ligand associated with a pathological condition, or having properties identified in the art as holding promise for treating a pathology. The scope of the term is intended to include all molecules independent of structural similarity or difference compared to the binding polypeptide so long as both can bind the same ligand. The molecule can be, for example, a naturally occurring molecule, a synthetic molecule, compound or macromolecule.

As used herein the term “changing” when used in reference to a parent polypeptide refers to modifying the structure of the parent polypeptide. Modification of the structure of a parent polypeptide can include, for example, adding a moiety, deleting an amino acid, substituting an amino acid or chemically modifying an amino acid. A moiety that can be substituted includes, for example, a chemically derivatized amino acid, D-stereoisomer of an amino acid, non-naturally occurring amino acid, amino acid analogue or mimetic of an amino acid. A chemical modification of an amino acid includes, for example, a covalent change in the bonding structure of an amino group at the alpha position, lysine, histidine, arginine, or tryptophan; covalent change in the bonding structure of a carbonyl at the alpha position, aspartate or glutamate or covalent change in the bonding structure of a sulfur at cysteine, cystine or methionine.

As used herein, the term “measuring,” when used in reference to an association rate, refers to a determination correlating the appearance of a species composed of a binding polypeptide bound to ligand with at least one defined time interval. Therefore, the term encompasses determination of an amount of time or rate at which a binding polypeptide binds to a ligand. Determination of association rate is meaningful when performed in a non-equilibrium state. Non-equilibrium states include, for example, pre-equilibrium, which can occur following mixture of free ligand with free binding polypeptide and post-equilibrium, which can occur following altering the concentration of species in an equilibrated mixture. Post-equilibrium determination of association rate includes, for example determination of k_{off} and using the value to calculate kₒₙ from Kₐ or K_{d} measured for the binding polypeptide and ligand.

Pre-equilibrium determination of association rate includes a relative determination, quantitative determination or time based selection. A relative determination includes a method involving comparing rates of association for two binding molecules under similar conditions such that quantitation of individual rates is not necessary. A quantitative determination includes a method for determining numerical value for an association rate or a rate constant such as kₒₙ. A time based selection includes, for example, exploiting a change in a property of a ligand or binding polypeptide that occurs when a binding polypeptide associates with ligand so as to select the bound species at a specified time interval.

A determination correlating the appearance of a species composed of a binding polypeptide bound to ligand involves a time dependent change, from a first state to a second state, for any property that changes when the binding polypeptide associates with ligand including, for example, absorption and emission of heat, absorption and emission of electromagnetic radiation, refractive index of surrounding solvent, affinity for a receptor, molecular weight, density, electric charge, polarity, molecular shape, or molecular size. A property that changes when a binding polypeptide associates with ligand can be transient, returning to the first state while the binding polypeptide is bound to ligand, or can remain in the second state the entire time that the binding polypeptide and ligand are bound.

As used herein, the term “identifying,” when used in reference to a binding polypeptide with an increased association rate, refers to recognizing a binding polypeptide as having an increased association rate. A binding polypeptide having increased association rate can be recognized prior to being isolated from a population, after being isolated from a population or the process of isolating the binding polypeptide from a population can be a form of recognizing a binding polypeptide with an increased association rate. A binding polypeptide having increased association rate can be recognized by comparing the association rate or kₒₙ value with an association rate or kₒₙ value for another binding molecule or by selecting a binding polypeptide based on a more rapid association rate. As such, recognizing a binding polypeptide with an improved association rate or kₒₙ can involve manual methods or automated methods.

As used herein the term “pathological condition” refers to a disease or abnormal condition including, for example, an injury of a mammalian cell or tissue. A pathological condition can be a disease or abnormal condition that results in unwanted or abnormal cell growth, viability or proliferation. A pathological condition characterized by unwanted or abnormal cell growth includes, for example, cancer or other neoplastic condition, infectious disease or autoimmune disease. For example, cancer cells proliferate in an unregulated manner and consequently result in tissue destruction. Similarly, the proliferation of cells mediating autoimmune diseases are aberrantly regulated which results in, for example, the continued, proliferation and activation of immune mechanisms with destruction of the host's cells and tissue. Specific examples of cancer include prostate, breast, lung, ovary, uterus, brain and skin cancer. Specific examples of infectious diseases include DNA or RNA viral diseases, bacterial diseases, parasitic diseases whereas autoimmune diseases include, for example, diabetes, rheumatoid arthritis and multiple sclerosis.

The invention provides a binding polypeptide, or functional fragment thereof, having a kₒₙ of at least about 9×10⁷ M⁻¹s⁻¹ for associating with a ligand and having therapeutic potency.

A binding polypeptide having therapeutic potency will demonstrate a therapeutic effect and exhibit expeditious association with a ligand to cure, alleviate, remove or lessen the symptoms of, or prevent or reduce the possibility of contracting a pathological condition. A binding polypeptide of the invention having therapeutic potency is understood to be a high potency binding polypeptide. Therapeutic potency can be identified in vitro according to a kinetic property, specifically, the association rate for binding polypeptide associating with a ligand. A binding polypeptide having therapeutic potency can be, for example, a binding polypeptide that prevents or reduces a pathological condition by associating with a ligand and preventing its binding to a receptor that is localized on a cell surface. A binding polypeptide having an increased association rate when associating with a ligand will have improved therapeutic potency compared to a polypeptide, including a binding polypeptide, that has a lower association rate when associating with the same ligand. Therefore, association rate indicates, and correlates with, therapeutic potency and, as such, provides a predictive measure of efficacy or relative efficacy.

A binding polypeptide can also be, for example, attached to a cytotoxic or cytostatic agent so as to deliver the agent to a cell experiencing a pathological condition by associating with a ligand localized on the surface of the cell. A binding polypeptide attached to a cytotoxic or cytostatic agent having an increased association rate when associating with the ligand will have improved therapeutic potency compared to a polypeptide that has a lower association rate when associating with the same ligand.

A binding polypeptide of the invention will be identified according to its ability to selectively associate with a ligand. Selective binding between a binding polypeptide and a ligand can be identified by methods known in the art. Methods of determining selective binding include, for example, equilibrium binding analysis, competition assays, and kinetic assays as described in Segel, Enzyme Kinetics John Wiley and Sons, New York (1975), which is incorporated herein by reference. Thermodynamic constants can be used to identify and compare binding polypeptides and ligands that selectively bind each other and include, for example, dissociation constant or K_{d}, association constant or Kₐ and Michaelis constant or Kₘ.

A binding polypeptide that can be used in the methods of the invention includes any polypeptide known to bind a ligand, made to bind a ligand, or known to be capable of binding a ligand. Therefore, a binding polypeptide of the invention can be selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

A binding polypeptide can be a naturally or non-naturally occurring polypeptide. A naturally occurring binding polypeptide can be obtained, for example, from a native tissue by directly isolating the polypeptide or by isolating the nucleotide encoding the polypeptide and expressing the polypeptide in a recombinant system. One skilled in the art can isolate the nucleotide encoding the polypeptide and express the polypeptide in a recombinant expression system according to methods known in the art as described, for example, in Goeddel, Methods in Enzymology, Vol 185, Academic Press, San Diego (1990); Wu, Methods in Enzymology, Vol 217, Academic Press, San Diego (1993); Sambrook et al., Molecular Cloning: A Laboratory Manual, Cold Spring Harbor Laboratory, New York (1992), and in Ausebel et al., Current protocols in Molecular Biology, John Wiley and Sons, Baltimore, Md. (2000), which are incorporated herein by reference. Methods of isolation of a parent polypeptide from recombinant and native tissues are well known in the art and are described, for example, in Scopes, Protein Purification: Principles and Practice, 3^{rd} Ed., Springer-Verlag, New York (1994); Duetscher, Methods in Enzymology, Vol 182, Academic Press, San Diego (1990), and Coligan et al., Current protocols in Protein Science, John Wiley and Sons, Baltimore, Md. (2000), which are incorporated herein by reference.

A naturally occurring binding polypeptide can be, for example, synthesized or produced in a recombinant expression system. For example, a binding polypeptide can be identified from a polypeptide sequence or a sequence of a nucleotide encoding a polypeptide isolated from a natural source or the nucleotide or polypeptide sequence can be obtained from a sequence data base including, for example, GenBank or other databases known in the art. Methods for isolating and sequencing nucleotides and polypeptides are well known in the art and are described, for example, in Sambrook et al., supra and in Ausubel et al., supra. A binding polypeptide can be expressed in a recombinant system using methods well known in the art including, for example, those described herein below. A binding polypeptide can also be produced by synthetic methods well known in the art, for example, Merrifield solid phase synthesis, t-Boc based synthesis, Fmoc synthesis and variations thereof.

A binding polypeptide of the invention can be non-naturally occurring. A non-naturally occurring polypeptide can be selected, for example, from a randomized population of polypeptides. A randomized population of non-naturally occurring polypeptides can be produced by peptide synthesis methods that are well known in the art including, for example, those described above. Methods of selecting a parent polypeptide from a population of polypeptides will be specific to the parent polypeptide to be selected, and can be achieved using methods well known by one skilled in the art based on the physical and chemical properties of the polypeptide.

A binding polypeptide of the invention can be a naturally occurring or non-naturally occurring polypeptide that is modified for use in the methods of the invention. A modification to facilitate use of a binding polypeptide in the methods of the invention can include, for example, incorporation of a label for detection of the polypeptide, incorporation of a binding group for capture of a binding polypeptide or modification to increase stability of the polypeptide. A label that can be incorporated includes, for example, a fluorophore, chromophore, paramagnetic spin label, or radionucleotide. A binding group that can be used to capture a polypeptide includes, for example, a biotin, polyhistidine tag (Qiagen; Chatsworth, Calif.), antibody epitope such as the flag peptide (Sigma; St Louis, Mo.), glutathione-S-transferase (Amersham pharmacia; Piscataway, N.J.), cellulose binding domain (Novagen; Madison, Wis.), calmodulin (Stratagene; San Diego, Calif.), staphylococcus protein A (Pharmacia; Uppsala, Sweden), maltose binding protein (New England BioLabs; Beverley, Mass.) or strep-tag (Genosys; Woodlands, Tex.) or minor modifications thereof. A modifications to increase stability can include, for example, incorporation of a cysteine to form a thioether crosslink, removal of a protease recognition sequence, addition of a charged amino acid to promote ionic interactions, or addition of a hydrophobic amino acid to promote hydrophobic interactions. The methods of the invention can accommodate other modifications that can confer additional properties onto the binding polypeptide of the invention so long as such modifications do not inhibit binding activity of the binding polypeptide. Examples include, addition of amino acids, deletion of amino acids, substitution of amino acids, chemical modification of amino acids and incorporation of non-natural amino acids.

A binding polypeptide of the invention is intended to include minor structural modifications that do not significantly change binding activity. For example, homologs or isotypes of a binding polypeptide can be isolated or synthesized that have minor structural modifications and similar binding activity when compared to the binding polypeptide and are included in the scope of a binding polypeptide of the invention. One skilled in the art can identify homologs or isotypes, for example, by aligning the sequences with an algorithm such as BLAST (Altschul et al., J. Mol. Biol. 215:403-410 (1990)), WU-BLAST2 (Altschull and Gish, Meth. Enzymol. 266:460-480 (1996)), FASTA (Pearson, Meth. Enzymol. 266:227-258 (1996)), or SSEARCH (Pearson, supra) to identify regions of structural homology. One skilled in the art can also identify homologues or isotypes using an algorithm that compares polypeptide structure including, for example, SCOP, CATH, or FSSP which are reviewed in Hadley and Jones Structure 7:1099-1112 (1999). The publications cited to reference sequence and structural alignment algorithms are incorporated herein. Site directed mutagenesis methods including, for example, those described herein, can be used to make the appropriate changes to modify homologous polypeptides to have similar association rate and therapeutic potency as a binding polypeptide of the invention. Differences between the homologous binding polypeptides having an insignificant effect on association rate and, therefore, therapeutic potency are considered to be minor modifications. For example, a second antibody from a second species can be modified to have similar association rate when associating with a ligand when compared to a first antibody from a first species that was produced or used in the methods of the invention.

Minor modifications that do not significantly change binding activity include, for example, a change made in a region of a binding polypeptide that does not affect the function of a region of the binding polypeptide that contacts ligand, conservative substitution of one or more amino acids that does not affect interactions between a binding polypeptide and ligand, and substitution of a functionally equivalent amino acid. A change made in the region that does not affect the function of a region of the binding polypeptide that contacts a ligand can include, for example, addition of one or more amino acid, addition of one or more moiety, deletion of one or more amino acid, substitution of one or more amino acid or chemical modification of one or more amino acid. A minor modification can be conservative substitution of an amino acid. Conservative substitutions of encoded amino acids can include, for example, amino acids which belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His). Other minor modifications are included so long as the binding polypeptide retains binding activity. The substitution of functionally equivalent amino acids is routine and can be accomplished by methods known to those skilled in the art. Briefly, the substitution of functionally equivalent amino acids can be made by identifying the amino acids which are desired to be changed, incorporating the changes into the encoding nucleic acid and then determining the function of the recombinantly expressed and modified binding polypeptide.

The invention also provides a grafted antibody, or functional fragment thereof, having a kₒₙ of at least about 1.3×10⁶ M⁻¹ s⁻¹ to a ligand and having therapeutic potency.

In one embodiment of the invention the binding polypeptide having therapeutic potency or high potency can be a grafted antibody. Grafted antibodies and methods for making grafted antibodies have been described herein previously. Accordingly, an antibody or functional fragment thereof can have human constant regions, or a heavy or light chain framework region at least a part of which is derived from one or more human antibody. A heavy or light chain framework regions used in an antibody or fragment can be derived from a particular antibody or from a consensus sequence of human antibodies. A grafted antibody having therapeutic potency can be produced or identified by the methods of the invention. An antibody or functional fragment thereof of the invention can be an antibody other than vitaxin.

The invention also provides a human antibody, or functional fragment thereof, comprising a kₒₙ of at least about 9×10⁷ M⁻¹s⁻¹ to a ligand and having therapeutic potency. Methods for identifying and producing human antibodies are well known in the art including, for example, those described in Harlow and Lane, supra.

An antibody or immunoglobulin of the invention can be a neutralizing antibody or neutralizing immunoglobulin. The term “neutralizing” refers to the ability to reduce the replication of microorganisms or viruses in an organism or in a cell that is cultured. Thus, an antibody or functional fragment thereof having therapeutic potency can have specificity for an antigenic determinant found on a microbe such as a virus, bacteria or fungus. Examples of viruses to which an antibody or fragment thereof can have specificity are respiratory syncytial virus or parainfluenza virus. A neutralizing antibody or neutralizing immunoglobulin the invention including active fragments thereof can be specific for at least one protein expressed by a virus such as RSV or PIV. A protein expressed by the RSV can be the F protein.

The invention provides a method of determining the therapeutic potency of a binding polypeptide. The methods consist of (a) contacting a binding polypeptide with a ligand; (b) measuring association rate for binding between the binding polypeptide and the ligand, and (c) comparing the association rate for the binding polypeptide to an association rate for a therapeutic control, the relative association rate for the binding polypeptide compared to the association rate for the therapeutic control indicating that the binding polypeptide will exhibit a difference in therapeutic potency correlative with the difference between the association rates. The invention further provides a method where the association rate is indicated by kₒₙ. For example, the kₒₙ for a binding polypeptide of the invention can be at least about 8×10⁶ M⁻¹s⁻¹. A binding polypeptide of the invention can also have a kₒₙ of at least about 9×10⁶ M⁻¹s⁻¹, 1×10⁷ M⁻¹s⁻¹, 2×10⁷ M⁻¹s⁻¹, 3×10⁷ M⁻¹s⁻¹, 4×10⁷ M⁻¹s⁻¹, 5×10⁷ M⁻¹s⁻¹, 6×10⁷ M⁻¹s⁻¹, 7×10⁷ M⁻¹s⁻¹, 8×10⁷ M⁻¹s⁻¹, 9×10⁷ M⁻¹s⁻¹, or 1×10⁸ M⁻¹s⁻¹ or higher. Binding polypeptides having lower kₒₙ can also have therapeutic potency. For example, a therapeutically potent polypeptide can have kₒₙ less than 8×10⁶ M⁻¹s⁻¹. Thus, binding polypeptides having kₒₙ of 1×10⁶ M⁻¹s⁻¹ can have therapeutic potency as can polypeptides having kₒₙ of 1×10⁴ M⁻¹s⁻¹. Accordingly, therapeutically potent polypeptides can have kₒₙ values of about 5×10⁴ M⁻¹s⁻¹, 1×10⁵ M⁻¹s⁻¹, 2.5×10⁵ M⁻¹ sec⁻¹, 5×10⁵ M⁻¹s⁻¹ or 7.5×10⁵ M⁻¹ sec⁻¹.

An association rate can be determined in any non-equilibrium mixture including, for example, one formed by rapidly contacting a binding polypeptide and ligand or by rapidly changing temperature. A non-equilibrium mixture can be a pre-equilibrium mixture. A pre-equilibrium mixture can be formed, for example, by contacting a soluble binding polypeptide and soluble ligand in a condition where the amount of total ligand and total binding polypeptide in the detection chamber are constant. Measurements of association rates in pre-equilibrium mixtures can be made in formats providing rapid mixing of binding polypeptide with ligand and rapid detection of changing properties of the binding polypeptide or ligand on a timescale of milliseconds or faster. Stopped flow and rapid quench flow instruments such as those described below provide a convenient means to measure non-equilibrium kinetics. The association rate can also be measured in non-equilibrium mixtures including, for example, solutions containing insoluble species of binding polypeptide, ligand or binding polypeptide bound to ligand, or solutions containing variable concentrations of total ligand or total binding polypeptide. Measurement of an association rate in a non-equilibrium mixture can be made in formats providing attachment of a ligand to a surface and continuous flow of a solution containing the binding polypeptide over the surface, or vice-versa, combined with rapid detection of changing properties of the binding polypeptide, ligand or surface such that measurements are made on a timescale of milliseconds or faster. Examples of formats providing non-equilibrium measurement of association rates include surface plasmon resonance instruments and evanescent wave instruments as described below.

Binding polypeptides and ligands to be contacted in mixtures for determination of association rate can be attached to another molecule, ligand or surface so long as they are capable of binding with their ligand or binding polypeptide partner respectively. Molecules that can be attached to a binding polypeptide or ligand include, for example, labels and binding groups such as those described herein previously for incorporation into binding polypeptide. Attached ligands can include, for example, an inhibitor that is competitively displaced when binding occurs between binding polypeptide and ligand, a second ligand that binds to the binding polypeptide such that binding can occur between the binding polypeptide and ligand of interest, or a second binding polypeptide that binds to ligand such that binding can occur between the binding polypeptide of interest and the ligand. Attached surfaces can include, for example, a dextran surface, polymer bead, biological membrane, or any biosensor surface.

Association rate measurements can be made by detecting the change in a property of the binding polypeptide or ligand that exists between the bound and unbound state or by detecting a change in the surrounding environment when binding polypeptide and ligand associate. Properties of the binding polypeptide or ligand that can change upon association and that can be used to measure association rates include, for example, absorption and emission of heat, absorption and emission of electromagnetic radiation, affinity for a receptor, molecular weight, density, mass, electric charge, conductivity, magnetic moment of nuclei, spin state of electrons, polarity, molecular shape, or molecular size. Properties of the surrounding environment that can change when binding polypeptide associates with ligand include, for example, temperature and refractive index of surrounding solvent.

Formats for measuring association rates in pre-equilibrium mixtures include, for example, stopped flow kinetic instruments and rapid quench flow instruments. A stopped flow instrument can be used to push solutions containing a binding polypeptide and ligand from separate reservoirs into a mixing chamber just prior to passage into a detection cell. The instrument can then detect a change in one or more of the above described properties to monitor progress of the binding event. A rapid quench flow instrument can be used to rapidly mix a solution containing a binding polypeptide with a solution containing a ligand followed by quenching the binding reaction after a finite amount of time. A change in one or more of the above described properties can then be detected for quenched mixtures produced by quenching at different times following mixing. Quenching can be performed for example by freezing or addition of a chemical quenching agent so long as the quenching step does not inhibit detection of the property relied upon for measurement of binding rate. Thus, a rapid quench instrument can be useful, for example, in situations where spectroscopic detection is not convenient. A variety of instruments are commercially available from vendors such as KinTek Corp. (State College, Pa.) and Hi-Tech Scientific (Salisbury, UK).

Formats for measuring association rates in non-equilibrium mixtures include, for example, surface plasmon resonance and evanescent wave instruments. Surface plasmon resonance and evanescent wave technology utilize a ligand or binding polypeptide attached to a biosensor surface and a solution containing either the binding polypeptide or ligand respectively that is passed over the biosensor surface. The change in refractive index of the solution that occurs at the surface of a chip when binding polypeptide associates with ligand can be measured in a time dependent fashion. For example, surface plasmon resonance is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (kₒₙ) and disassociation rates (k_{off}). Surface plasmon resonance instruments are available in the art including, for example, the BIAcore instrument, IBIS system, SPR-CELLIA system, Spreeta, and Plasmon SPR and evanescent wave technology is available in the Iasys system as described, for example, in Rich and Myszka, Curr. Opin. Biotech. 11:54-61 (2000).

The association rate can be determined by measuring a change in a property of a ligand or binding polypeptide at one or more discreet time intervals during the binding event using, for example, the methods described above. Measurements determined at discreet time intervals during the binding event can be used to determine a quantitative measure of association rate or a relative measure of association rate. Quantitative measures of association rate can include, for example, an association rate value or kₒₙ value. Quantitative values of association rate or kₒₙ can be determined from a mathematical or graphical analysis of a time dependent measurement. Such analyses are well known in the art and include algorithms for fitting data to a sum of exponential or linear terms or algorithms for computer simulation to fit data to a binding model as described for example in Johnson, Cur. Opin. Biotech. 9:87-89 (1998), which is incorporated herein by reference.

Association rates can be determined from mixtures containing insoluble species or variable concentrations of total ligand or total binding polypeptide using mathematical and graphical analyses such as those described above if effects of mass transport are accounted for in the reaction. One skilled in the art can account for mass transport by comparing association rates under conditions having similar limitations with respect to mass transport or by adjusting the calculated association rate according to models available in the art including, for example those described in Myszka et al., Biophys. J. 75:583-594 (1998), which is incorporated herein by reference.

A higher value of either the association rate or kₒₙ is indicative of improved therapeutic potency. Thus, quantitative determinations provide an advantage by allowing comparison between an association rate of a binding polypeptide and a therapeutic control determined by different methods so long as the methods used are understood by one skilled in the art to yield consistent results.

A relative measure of association rate can include, for example, comparison of association rate for two or more binding polypeptides binding to ligand under similar conditions or comparison of association rate for a binding polypeptide binding to ligand with a predefined rate. Comparison of association rate for two or more binding polypeptides can include a standard of known association rate or a molecule of known therapeutic effect. A predefined rate used for comparison can be determined by calibrating the measurement to be relative to a previously measured rate including, for example, one available in the scientific literature or in a database. An example of a comparison with a predefined rate is selection of the species of binding polypeptide bound to ligand at a discreet time interval defined by the predefined rate by using a time actuated selection device.

An advantage of the invention is that the methods can be used with any ligand that mediates or specifically correlates with a pathological condition. The methods can also be used with a structurally modified adduct of a ligand that mediates or specifically correlates with a pathological condition, or a ligand that mimics binding function of a ligand that mediates or specifically correlates with a pathological condition. Structural modifications can facilitate use of a ligand in the methods of the invention and can include, for example, incorporation of labels for detection of the ligand, incorporation of binding groups for capture of the ligand or modifications to increase stability of the ligand. Labels, binding groups and modifications to increase stability include, for example, those described herein previously for incorporation into polypeptides. It can also be advantageous to use a mimic of the ligand to bias the binding interaction with respect to a subset of physical interactions that influence its functional association with a binding polypeptide. Physical interactions that allow a ligand and binding polypeptide to associate include, for example, hydrogen bonds, ionic forces, van der Waals interactions or hydrophobic interactions or a combination thereof.

A ligand used with the methods of the invention can be synthesized or isolated from a natural source by a variety of methods known in the art. Synthetic methods for synthesizing a ligand include, for example, organic synthesis, cell free synthesis using extracted cellular components, and chemical synthesis. A ligand that is a polypeptide or nucleic acid can be synthesized, for example, in a recombinant expression system using methods similar to those described below. Additionally, a ligand can be produced in a recombinant organism modified to express one or more enzymes that convert a host intermediate or exogenously supplied intermediate into the ligand. Isolation of a ligand from a natural source can be performed by methods known in the art. For example, a polypeptide or nucleic acid based ligand can be isolated as described herein for a parent polypeptide or binding polypeptide and their encoding nucleic acids. Small molecule ligands can be isolated according to methods known in the art including, for example, extraction, chromatography, crystallization or distillation. Methods of isolating small molecules can be found, for example, in Gordon and Ford, The Chemist's Companion, John Wiley and Sons (1973) and Vogel, Vogel's Textbook of Practical Organic Chemistry, 5^{th} Ed., Addison-Wesley Pub. Co. (1989).

Binding polypeptides having improved therapeutic potency can be determined or identified by comparing an association rate for binding between a binding polypeptide and ligand with an association rate for a therapeutic control binding to the ligand. Since the therapeutic potency of the therapeutic control is correlated with its association rate for associating with a ligand, the therapeutic control provides a means of determining therapeutic potency according to association rates measured in vitro.

A therapeutic control can be any molecule so long as the molecule associates with the same ligand as the binding polypeptide to be compared. The therapeutic control of the invention can include, for example, a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein, clotting factor, or functional fragments thereof.

For purposes of comparison, the association rate of a binding polypeptide and ligand can be determined relative to association rate for a therapeutic control and the same ligand. A comparison can also be made according to a quantitative association rate for binding polypeptide and ligand compared to a quantitative association rate for a therapeutic control and ligand. Relative or quantitative association rates can be determined by the methods described above. Determination of association rates for a binding polypeptide associating with a ligand can be performed simultaneously with a binding polypeptide and therapeutic control or at separate times provided conditions are sufficiently similar in each assay to allow valid comparison. Thus, association rate determined for a binding polypeptide by the methods of the invention can be compared to a previously measured association rate for a therapeutic control.

The invention provides a method of determining the therapeutic potency of a binding polypeptide. The method consists of (a) contacting a binding polypeptide with a ligand; (b) measuring association rate for binding between the binding polypeptide and the ligand; (c) comparing the association rate for the binding polypeptide to an association rate for a therapeutic control, the relative association rate for the binding polypeptide compared to the association rate for the therapeutic control indicating that the binding polypeptide will exhibit a difference in therapeutic potency correlative with the difference between the association rates, and (d) changing one or more amino acids in the binding polypeptide and repeating steps (a) through (c) one or more times. In addition, steps (a) through (d) can be repeated one or more times and stopped at step (c). Increased association rate correlates with improved therapeutic potency where increases in association rate can be at least 4-fold, 5-fold, 6-fold, 7-fold, 8-fold, 9-fold or 10-fold or more.

Steps (a) through (c), as recited above, can be performed according to methods described herein previously for determining therapeutic potency of a binding polypeptide by measuring association rate. Step (d), recited above, provides an advantage in allowing one skilled in the art to use the methods of the invention to change the therapeutic potency of a binding polypeptide by changing the binding polypeptide and identifying a difference in therapeutic potency of the changed binding polypeptide from an association rate. Therapeutic potency of a binding polypeptide can be altered by changing the binding polypeptide to have an increased or decreased association rate when binding a ligand or to have an increased association rate when binding a new ligand. A binding polypeptide changed by the methods of the invention to have improved therapeutic potency by binding to a new ligand can have substantially unaltered association rate for the original ligand or can have an increase or decrease in association rate for the original ligand. Binding of a new ligand to a changed binding polypeptide can be competitive with binding of the original ligand, non-competitive with binding of the original ligand, or allosteric with binding of the original ligand. Competitive, non competitive and allosteric binding of two ligands to a binding polypeptide can be recognized by methods available in the art as described in Segel, supra.

Amino acids to be changed in a polypeptide in order to change therapeutic potency can be incorporated randomly or incorporated based on knowledge of the interactions between the binding polypeptide and ligand. Random incorporation includes, for example, incorporating each of the twenty naturally occurring amino acid residues, or a subset thereof, at one or more defined position or incorporating each of the twenty naturally occurring amino acid residues, or a subset thereof, at random positions in the polypeptide or portion thereof. For example, a portion of a polypeptide can be randomly changed to incorporate all 20 natural amino acids or a subset thereof. As an example of changing random sites in a polypeptide, a polypeptide can be randomly mutated along its entire sequence by incorporating all 20 natural amino acids or a subset thereof.

Knowledge of interactions between a binding polypeptide and ligand can be used to guide site directed changes, to bias random changes, or to produce biased changes. For example, if residues of the original binding polypeptide are known to interact with a ligand these residues can be altered to accommodate or invoke interactions with a second ligand at the same site. Knowledge of the interactions between binding polypeptide and ligand can include, for example, identification of residues in the binding polypeptide that interact with the ligand, identification of residues that affect the structure or function of the binding polypeptide binding site or identification of residues that are proximal to the binding polypeptide ligand binding site. Such interactions can be derived from information on the structure and function of the binding polypeptide, ligand or binding polypeptide bound to ligand.

Structure and function information can be used to identify interactions between a binding polypeptide and ligand. For example, interactions can be identified from a structural model, amino acid sequence, functional binding data, or identification of sites or regions labeled with reagents that selectively modify amino acids of a binding polypeptide. Structural models of a binding polypeptide can be derived from, for example, X-ray crystallography, nuclear magnetic resonance spectroscopy, electron microscopy, atomic force microscopy, X-ray scattering or neutron scattering. A structural model can include structure of a binding polypeptide, structure of a ligand or structures of both a binding polypeptide and bound ligand. Molecular modeling can be used in conjunction with a structural model to identify potential interactions between a binding polypeptide and ligand.

The amino acid sequence of a binding polypeptide or ligand can be used, for example, to determine binding residues according to homology with other binding polypeptides and ligands. For example, amino acids to be changed in a first binding polypeptide can be chosen based on homology to amino acids known to interact with a ligand in a second polypeptide. Again molecular modeling can be used in conjunction with a homology search to model a putative structure for the binding polypeptide or ligand thereby allowing identification of potential interacting amino acids.

Functional binding studies with modified binding polypeptides can be useful in identifying regions to change in the methods of the invention. For example, a change in binding activity that correlates with a change in an amino acid of a binding polypeptide can indicate that the changed amino acid position potentially interacts with a ligand.

The size of a population of polypeptides produced from a randomly changed polypeptide can be minimized by introducing a bias into random mutagenesis methods. A bias can be introduced with respect to the particular amino acids to be incorporated, with respect to the amino acid sites at which a polypeptide is changed, or with respect to both the particular amino acid to be incorporated and the site of incorporation.

A bias can also be introduced into the randomization at a specified position based on conservative substitutions. Conservative substitutions of amino acids include, for example, (1) non-polar amino acids (Gly, Ala, Val, Leu and Ile); (2) polar neutral amino acids (Cys, Met, Ser, Thr, Asn and Gln); (3) polar acidic amino acids (Asp and Glu); (4) polar basic amino acids (Lys, Arg and His); and (5) aromatic amino acids (Phe, Tyr, Trp and His). Additionally, conservative substitutions of amino acids include, for example, substitutions based on the frequencies of amino acid changes between corresponding proteins of homologous organisms as described, for example, in Principles of Protein Structure, Schulz and Schirmer, eds., Springer Verlag, New York (1979) which is incorporated herein by reference.

A subset of residues for randomization within a polypeptide can be chosen based on properties of the polypeptide. For example, biased mutagenesis of proteases, protease inhibitors, immunoglobulins, DNA binding polypeptides and RNA binding polypeptides is described in Methods in Enzymology 267:52-68 (1996), biased mutagenesis of streptavidin is described in Voss and Skerra, Prot. Eng. 10:975-982 (1997), biased mutagenesis of binding polypeptides having a lipocalin fold is described in Beste et al. Proc Natl. Acad. Sci. USA 96:1898-1903 (1999), biased mutagenesis of growth hormones is described in Ballinger et al., J. Biol. Chem. 273:11675-11684 (1998) and biased mutagenesis of an antibody is described in Wu et al., Proc. Natl. Acad. Sci. USA 95:6037-6042 (1998).

Random mutagenesis and biased mutagenesis methods can produce changes at one or more selected positions without altering the remaining amino acid positions within a region. For example, a population of single position changes can contain varied amino acid residues at each position, incorporated either randomly or with a biased frequency, while leaving the remaining positions unchanged. For the specific example of a ten residue region, a population can contain species having the first, second and third, continued through the tenth residue, independently randomized or represented by a biased frequency of incorporated amino acid residues while keeping the remaining positions unchanged. For the specific example described above, these non-varied positions would correspond to positions 2-10; 1,3-10; 1,2,4-10, continued through positions 1-9, respectively. Therefore, the resultant population will contain species that represent all single position changes.

Similarly, double, triple quadruple or more amino acid position changes can be generated within a region of a polypeptide without altering the remaining amino acid positions. For example, a population of double position changes will contain at each set of two positions the varied amino acid residues while leaving the remaining positions as unchanged residues. The sets will correspond to, for example, positions 1 and 2, 1 and 3, 1 and 4, and continued pairwise through the region until the last set corresponds to the first and last positions of the region. The population will also contain sets corresponding to positions 2 and 3, 2 and 4, 2 and 5, through the set corresponding to the second and last position of the region. Similarly, the population will contain sets of double position changes corresponding to all pairs of position changes beginning with position three of the region. Similar pairs of position changes are made with the remaining sets of amino acid positions. Therefore, the population will contain species that represent all pairwise combinations of amino acid position changes. In a similar fashion, populations corresponding to sets of changes representing all triple and quadruplet changes along a region can similarly be targeted using the methods of the invention.

Because the methods of the invention can employ the production and screening of diverse populations of polypeptides, effects on association rate, such as the neutralization or augmentation of inherently detrimental changes and the neutralization or augmentation of beneficial amino acid changes, can occur due to the combined interactions of two or more amino acid changes within a single polypeptide. No prior information is required to assess which amino acid positions or changes can be inherently beneficial or detrimental, or which positions or changes can be further augmented by second site changes. Instead, by selecting amino acid positions or subsets thereof and generating a diverse population containing amino acid variants at these positions, combinations of beneficial changes occurring at the selected positions will be identified by screening for increased or optimized association rate. Such beneficial combinations will include the unveiling of inherently beneficial residues and neutralization of inherently detrimental residues.

Methods for efficient synthesis and expression of populations of changed polypeptides synthesized using oligonucleotide-directed mutagenesis can be performed, for example, as previously described in Wu et al. supra; Wu et al., J. Mol. Biol., 294:151-162 (1999) and Kunkel, Proc. Natl. Acad. Sci. USA, 82:488-492 (1985) which are incorporated herein by reference. Oligonucleotide-directed mutagenesis is a well known and efficient procedure for systematically introducing mutations, independent of their phenotype and is, therefore, suited for directed evolution approaches to protein engineering. The methodology is flexible, permitting precise mutations to be introduced without the use of restriction enzymes, and is relatively inexpensive. Briefly, to perform oligonucleotide directed mutagenesis, a population of oligonucleotides encoding the desired mutation(s) is hybridized to single-stranded uracil-containing template of the wild type sequence, double-stranded circular DNA is generated by a polymerase and a ligase, and the mutant DNA is efficiently recovered following transformation of a dut⁺ ung⁺ bacterial strain which can not replicate the uracil containing wild-type template.

Populations of changed polypeptides can also be generated using gene shuffling. Gene shuffling or DNA shuffling is a method for directed evolution that generates diversity by recombination as described, for example, in Stemmer, Proc. Natl. Acad. Sci. USA 91:10747-10751 (1994); Stemmer, Nature 370:389-391 (1994); Crameri et al., Nature 391:288-291 (1998); Stemmer et al., U.S. Pat. No. 5,830,721, which are incorporated herein by reference. Gene shuffling or DNA shuffling is a method using in vitro homologous recombination of pools of selected mutant genes. For example, a pool of point mutants of a particular gene can be used. The genes are randomly fragmented, for example, using DNase, and reassembled by PCR. If desired, DNA shuffling can be carried out using homologous genes from different organisms to generate diversity (Crameri et al., supra, 1998). The fragmentation and reassembly can be carried out, for example, in multiple rounds, if desired. The resulting reassembled genes are a population of variants that can be used in the invention.

Simultaneous incorporation of all of the encoding nucleic acids and all of the selected amino acid position changes can be accomplished by a variety of methods known to those skilled in the art, including for example, recombinant and chemical synthesis. Simultaneous incorporation can be accomplished by, for example, chemically synthesizing the nucleotide sequence for the region and incorporating at the positions selected for harboring variable amino acid residues a plurality of corresponding amino acid codons.

One method well known in the art for rapidly and efficiently producing a large number of alterations in a known amino acid sequence or for generating a diverse population of variable or random sequences is known as codon-based synthesis or mutagenesis. This method is the subject matter of U.S. Pat. Nos. 5,264,563 and 5,523,388 and is also described in Glaser et al. J. Immunology 149:3903 (1992), all of which are incorporated herein by reference. Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. For the synthesis of equal frequencies of all twenty amino acids, up to two codons can be synthesized in each reaction vessel.

Variations to this synthesis method also exist and include, for example, the synthesis of predetermined codons at desired positions and the biased synthesis of a predetermined sequence at one or more codon positions. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of NNG/T nucleotides where N is a mixture of all four nucleotides. Following synthesis of the predetermined and random codons, the reaction products in each of the two reaction vessels are mixed and then redivided into an additional two vessels for synthesis at the next codon position.

A modification to the above-described codon-based synthesis for producing a diverse number of changed sequences can similarly be employed for the production of changed polypeptide populations described herein. This modification is based on the two vessel method described above which biases synthesis toward the parent sequence and allows the user to separate the variants into populations containing a specified number of codon positions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds by then dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three and four positions as well as others can be conveniently separated out and used based on the need of the individual. Moreover, this synthesis scheme also allows enrichment of the populations for the randomized sequences over the parent sequence since the vessel containing only the parent sequence synthesis is similarly separated out from the random codon synthesis.

Other methods well known in the art for producing a large number of alterations in a known amino acid sequence or for generating a diverse population of variable or random sequences include, for example, degenerate or partially degenerate oligonucleotide synthesis. Codons specifying equal mixtures of all four nucleotide monomers, represented as NNN, results in degenerate synthesis. Whereas partially degenerate synthesis can be accomplished using, for example, the NNG/T codon described previously. Other methods well known in the art can alternatively be used such as the use of statistically predetermined, or variegated, codon synthesis which is the subject matter of U.S. Pat. Nos. 5,223,409 and 5,403,484, which are incorporated herein by reference.

Once the populations of changed polypeptides encoding nucleic acids have been constructed as described above, they can be expressed to generate a population of changed polypeptides that can be screened for association rate. For example, the nucleic acids encoding the changed polypeptides can be cloned into an appropriate vector for propagation, manipulation and expression. Such vectors are known or can be constructed by those skilled in the art and should contain all expression elements sufficient for the transcription, translation, regulation, and if desired, sorting and secretion of the altered polypeptide or polypeptides. The vectors also can be for use in either procaryotic or eukaryotic host systems so long as the expression and regulatory elements are of compatible origin. The expression vectors can additionally included regulatory elements for inducible or cell type-specific expression. One skilled in the art will know which host systems are compatible with a particular vector and which regulatory or functional elements are sufficient to achieve expression of a polypeptide in soluble, secreted or cell surface forms.

Suitable expression vectors are well-known in the art and include vectors capable of expressing nucleic acid operatively linked to a regulatory sequence or element such as a promoter region or enhancer region that is capable of regulating expression of such nucleic acid. Promoters or enhancers, depending upon the nature of the regulation, can be constitutive or inducible. The regulatory sequences or regulatory elements are operatively linked to a nucleic acid of the invention or population of changed nucleic acids as described above in an appropriate orientation to allow transcription of the nucleic acid.

Appropriate expression vectors include those that are replicable in eukaryotic cells and/or prokaryotic cells and those that remain episomal or those which integrate into the host cell genome. Suitable vectors for expression in prokaryotic or eukaryotic cells are well known to those skilled in the art as described, for example, in Ausubel et al., supra. Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector useful in the methods of the invention can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell.

Appropriate host cells, include for example, bacteria and corresponding bacteriophage expression systems, yeast, avian, insect and mammalian cells and compatible expression systems known in the art corresponding to each host species. Methods for recombinant expression of populations of progeny polypeptides or progeny polypeptides within such populations in various host systems are well known in the art and are described, for example, in Sambrook et al., supra and in Ansubel et al., supra. The choice of a particular vector and host system for expression and screening of progeny polypeptides will be known by those skilled in the art and will depend on the preference of the user. Expression of diverse populations of hetereomeric receptors in either soluble or cell surface form using filamentous bacteriophage vector/host systems is well known in the art and is the subject matter of U.S. Pat. No. 5,871,974 which are incorporated herein by reference.

The recombinant cells are generated by introducing into a host cell a vector or population of vectors containing a nucleic acid molecule encoding a binding polypeptide. The recombinant cells are transducted, transfected or otherwise genetically modified by any of a variety of methods known in the art to incorporate exogenous nucleic acids into a cell or its genome. Exemplary host cells that can be used to express a binding polypeptide include mammalian primary cells; established mammalian cell lines, such as COS, CHO, HeLa, NIH3T3, HEK 293 and PC12 cells; amphibian cells, such as Xenopus embryos and oocytes; and other vertebrate cells. Exemplary host cells also include insect cells such as Drosophila, yeast cells such as Saccharomyces cerevisiae, Saccharomyces pombe, or Pichia pastoris, and prokaryotic cells such as Escherichia coli.

In one embodiment, a nucleic acids encoding a polypeptide can be delivered into mammalian cells, either in vivo or in vitro using suitable vectors well-known in the art. Suitable vectors for delivering a nucleic acid encoding a polypeptide to a mammalian cell, include viral vectors such as retroviral vectors, adenovirus, adeno-associated virus, lentivirus, herpesvirus, as well as non-viral vectors such as plasmid vectors.

Viral based systems provide the advantage of being able to introduce relatively high levels of the heterologous nucleic acid into a variety of cells. Suitable viral vectors for introducing a nucleic acid encoding a polypeptide into mammalian cells are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)). The above publications describing vectors or their use are incorporated herein by reference.

In addition to mutagenesis methods described above, a polypeptide can be changed by chemical modifications. Chemical modifications can be made to change the binding properties of a polypeptide, to benefit measurement of association rates or to benefit identification of a binding polypeptide. A chemical modification of an amino acid includes, for example, modification of amino groups by amidination, guanidination, reductive methylation, carbamylation, acetylation, trinitrobenzoylation, succinylation or formylation; modification of arginine by butanedione reaction, phenylglyoxal reaction, or nitromalondialdehyde reaction; modification of carbonyls by esterification or carbodiimide coupling; sulfenylation of tryptophan; modification of tyrosine by nitration or iodination; modification of sulfhydrils by reduction, oxidation, carboxymethylation, carboxyerthylation, aminoethylation, methylation, sulphonation, addition of thiols, or cyanylation. One skilled in the art can chemically modify a parent polypeptide by methods described in Means and Feeney, Chemical Modification of Proteins Holden-Day Inc., San Francisco (1971) and Glazer et al., Chemical Modification of Proteins: Selected methods and analytical procedures Elsevier Biomedical Press, New York (1975) which are incorporated herein by reference.

Changing the structure of a binding polypeptide by the methods described herein provides a means to alter therapeutic potency by increasing or decreasing the association rate or by increasing the association rate for a new ligand. Therefore, binding polypeptides having increased or decreased therapeutic potency can be identified according to the needs of the practitioner. The methods of the invention provide for production of a population of progeny polypeptides that has sufficient size and diversity to yield a likely probability of obtaining a binding polypeptide having desired changes in therapeutic potency, whether it be an increases or decrease. As described previously, the size and diversity of the population can be adjusted according to the chosen method of mutagenesis. For example, if random mutagenesis methods are to be employed then a large population of high diversity can be produced. The size or diversity of the population can be reduced by using biased mutagenesis, focused mutagenesis or site directed mutagenesis. One skilled in the art will be able to determine the size and diversity of the population of progeny polypeptides based on the properties of the particular polypeptide to be changed and which method is to be used for changing the polypeptide.

The methods of the invention provide for repetition of steps to further optimize the therapeutic potency of a binding polypeptide. The therapeutic potency of a binding polypeptide can be optimized by isolating a binding polypeptide having altered therapeutic potency and repeating the steps of the method described herein. Specifically, the therapeutic potency of the isolated binding polypeptide having altered therapeutic potency can be determined by changing one or more amino acid, contacting the isolated binding polypeptide having altered therapeutic potency with a ligand, measuring association rate for binding between the isolated binding polypeptide having altered therapeutic potency and the ligand, and comparing the association rate for the binding polypeptide to an association rate for a therapeutic control. The steps can be repeated once, twice, or many times until a desired therapeutic potency is obtained.

An example of a binding polypeptide that can be made and used with the methods of the invention is an antibody, or functional fragment thereof. For example, often grafted antibodies are observed to have reduced affinity when compared to the donor antibody from which the CDRs were derived. The methods of the invention can be used to improve the association rate for a grafted antibody binding to a ligand and, therefore, therapeutic potency of the grafted antibody. The grafted antibody binding site can be identified by any or all of the criteria specified previously and in the examples and the methods of the invention described previously with respect to binding polypeptides can be utilized. A grafted antibody can have at least 1, 2, 3, 4, 5 or 6 CDRs from a therapeutically potent antibody or other functional antibody. A CDR of an antibody or fragment thereof can be selected from a light chain CDR such as L1, L2 or L3 or heavy chain CDR such as H1, H2 and H3.

The invention further provides a method to determine therapeutic potency of a binding polypeptide where the difference between the kₒₙ for a binding polypeptide and the kₒₙ for a therapeutic control is independent of an effect of a difference between Kₐ for the binding polypeptide and Kₐ for the therapeutic control. Also provided is a method where the difference between the kₒₙ for the binding polypeptide and the kₒₙ for the therapeutic control can be an increase and Kₐ for the binding polypeptide can be a similar value to Kₐ for the therapeutic control. Similarly, a method is provided where the difference between the kₒₙ for the binding polypeptide and the kₒₙ for the therapeutic control can be an increase and Kₐ for the binding polypeptide can be a lower value than Kₐ for the therapeutic control.

An advantage of the invention is that a binding polypeptide having improved therapeutic potency can be distinguished from a binding polypeptide that has an increased Kₐ for a ligand but not improved therapeutic potency. Methods for identifying a therapeutic binding polypeptide based on Kₐ rely on an equilibrium measurement which, absent time dependent measurements made in a non-equilibrium condition, are inaccurate for identifying a binding polypeptide having increased association rate and therefore improved therapeutic potency. According to the relationship Kₐ=kₒₙ/k_{off}, an increased Kₐ for association of a binding polypeptide and ligand can be due to changes in kₒₙ or k_{off}. For example, a binding polypeptide having improved therapeutic potency can have a reduced Kₐ if a reduction in k_{off} occurs that over compensates for an increase in kₒₙ. Thus, changes in Kₐ, being influenced by changes in k_{off}, do not unambiguously correlate with changes in therapeutic potency since binding polypeptides having improved therapeutic potency can display either reduced or increased Kₐ.

A binding polypeptide having therapeutic potency such as an antibody or functional fragment thereof can have a Kₐ of at least about 1×10⁹ M⁻¹, 1×10¹⁰ M⁻¹ or 1×10¹¹ M⁻¹. A binding polypeptide of the invention such as an antibody or functional fragment thereof can be evaluated by other known measures such as EC₅₀. A binding polypeptide can have an EC₅₀ of less than about 6.0 nM, 3.0 nM, or 1.0 nM.

The invention provides a method of determining therapeutic potency of a binding polypeptide. The method consists of (a) contacting two or more binding polypeptides with a ligand; (b) measuring kₒₙ for binding between the two or more binding polypeptides and the ligand, and (c) identifying a binding polypeptide exhibiting a high kₒₙ, the kₒₙ value correlating with the therapeutic potency of the identified binding polypeptide.

The invention further provides a method of determining therapeutic potency of a binding polypeptide where the method consists of (a) contacting two or more binding polypeptides of a population with a ligand; (b) measuring association rates for the two or more binding polypeptides binding to the ligand; (c) comparing the association rates for the two or more binding polypeptides binding to the ligand, and (d) identifying a binding polypeptide exhibiting a higher association rate for binding to said ligand than one or more other binding polypeptides of the population, said higher association rate correlating with the therapeutic potency of said identified binding polypeptide. The association rate identified by the method can be indicated by kₒₙ. The kₒₙ of a binding polypeptide exhibiting a higher association rate for a ligand can be at least about 1.5×10⁶ M⁻¹. A binding polypeptide exhibiting a higher association rate for a ligand can also have a kₒₙ of at least about 2×10⁶ M⁻¹s⁻¹, 4×10⁶ M⁻¹s⁻¹, 6×10⁶ M⁻¹s⁻¹, 8×10⁶ M⁻¹s⁻¹, 1×10⁷ M⁻¹s⁻¹, 2×10⁷ M⁻¹s⁻¹, 4×10⁷ M⁻¹s⁻¹, 6×10⁷ M⁻¹s⁻¹, 8×10⁷ M⁻¹s⁻¹, or 1×10⁸ M⁻¹s⁻¹ or higher. Preferably, a high kₒₙ is larger than kₒₙ for a therapeutic control.

The step of contacting two or more binding polypeptides of a population with a ligand can be performed with binding polypeptides isolated from the population prior to being contacted with the ligand or a mixture containing two or more binding polypeptides from the population. The step of measuring association rates for a binding polypeptide isolated from the population can be performed according to essentially any of the methods described herein previously. Measuring association rates for binding polypeptides in a mixture containing two or more binding polypeptides can be performed by relative methods including, for example, selection of a binding polypeptide bound to ligand at a discreet time interval by using a time actuated collection device.

Comparing the association rates for two or more binding polypeptides isolated from a population can be achieved essentially as described previously. Comparing the association rates for two or more binding polypeptides in the same mixture can be achieved by selection methods, for example, using a time actuated device as described above. Such methods of comparison can be made with a population of binding polypeptides containing one or more binding polypeptide that are standards of known association rate or therapeutic potency. Additionally a population containing binding polypeptides of unknown association rate can be measured such that one or more binding polypeptides is identified as having increased association rate and improved therapeutic potency compared to the average for the population.

A population of polypeptides used in the methods of the invention can include 2, 10, 100, 1×10³, 1×10⁴, 1×10⁵, 1×10⁶, 1×10⁷, 1×10⁸, 1×10⁹, or 1×10¹⁰ or more different binding polypeptides. As described previously one skilled in the art will be able to determine the size and diversity of the population of binding polypeptides based on the properties of the particular polypeptide to be changed and which method is used to change the polypeptide. One skilled in the art can also alter the number of binding polypeptides to be measured from a population such that a sub-population can be measured. The number of polypeptides to be measured can be based on factors such as the diversity of the population, the magnitude of change desired in the therapeutic potency, or the degree of bias incorporated during mutagenesis. Accordingly, association rates can be measured for 2, 10, 100, 1×10³, 1×10⁴, 1×10⁵, 1×10⁶, 1×10⁷, 1×10⁸, 1×10⁹, or 1×10¹⁰ or more different binding polypeptides from a population.

The invention provides a method for producing one or more binding polypeptides with improved therapeutic potency. The method consists of (a) changing one or more amino acids in a parent polypeptide to produce one or more different progeny polypeptides; (b) measuring the association rate for the one or more different progeny polypeptides associating with a ligand, and (c) identifying a binding polypeptide from one or more progeny polypeptides having at least a 4-fold increase in association rate to a ligand compared to the parent polypeptide, the increased association rate resulting in improved therapeutic potency toward a pathological condition. Further provided is a method where the fold increase in association rate is indicated by an increase in kₒₙ. Therefore, kₒₙ can increase by 4-fold, 5-fold, 6-fold, 7-fold, 8-fold, 9-fold, or 10-fold or more in the methods of the invention. The increased kₒₙ can be at least about 3×10⁵ M⁻¹s⁻¹. The increased kₒₙ can also be at least about 5×10⁵ M⁻¹s⁻¹, 7×10⁵ M⁻¹s⁻¹, 9×10⁵ M⁻¹s⁻¹, 1×10⁶ M⁻¹s⁻¹, 3×10⁶ M⁻¹s⁻¹, 5×10⁶ M⁻¹s⁻¹, 7×10⁶ M⁻¹s⁻¹, 9×10⁶ M⁻¹s⁻¹ or 1×10⁷ M⁻¹s⁻¹ or more. Furthermore, the increase in kₒₙ resulting in improved therapeutic potency can be independent of an effect of a change in Kₐ for the binding polypeptide. The binding polypeptide having an increase in kₒₙ can have a Kₐ value similar to Kₐ for its parent polypeptide or a Kₐ value lower than Kₐ for its parent polypeptide.

A polypeptide changed by the methods of the invention can be a parent polypeptide. A parent polypeptide is one example of a peptide described herein and therefore can have any of the properties thereof and be made and used according to the description provided herein. For example, one or more amino acids in a parent polypeptide can be changed according to the previously described methods to produce one or more different progeny polypeptides. A progeny polypeptide is one example of the changed polypeptides described herein and can therefore be made and used according to the previous descriptions herein. Accordingly, the step of measuring an association rate for one or more different progeny polypeptides associating with a ligand, can be performed as described herein previously. In addition, the step of identifying a binding polypeptide from one or more progeny polypeptides having at least a 4-fold increase in association rate when binding to a ligand compared to its parent polypeptide can be performed according to the methods described previously herein for determining association rates and therapeutic potency.

The step of identifying a binding polypeptide from one or more progeny polypeptides having at least a 4-fold increase in association rate to a ligand compared to the parent polypeptide can be performed to identify a binding polypeptide from one or more progeny polypeptides having at least a 5-fold, 6-fold, 7-fold, 8-fold, 9-fold, or 10-fold or greater increase in association rate resulting in improved therapeutic potency toward a pathological condition. Binding polypeptides having a larger fold increase in association rate will have an increased therapeutic potency. Additionally the increased kₒₙ can be at least about 1×10⁸ M⁻¹s⁻¹, 1.5×10⁸ M⁻¹s⁻¹, 2×10⁸ M⁻¹s⁻¹, 2.5×10⁸ M⁻¹s⁻¹, 3×10⁸ M⁻¹s⁻¹, 4×10⁸ M⁻¹s⁻¹, 5×10⁸ M⁻¹s⁻¹, or 1×10⁹ M⁻¹s⁻¹ or more.

Therefore, the invention further provides a method for producing a binding polypeptide with improved therapeutic potency. The method consists of (a) changing one or more amino acids in a parent polypeptide to produce one or more different progeny polypeptides; (b) measuring the association rate for the one or more different progeny polypeptides associating with a ligand, and (c) identifying a binding polypeptide from the one or more different progeny polypeptides having a kₒₙ of at least about 1.5×10⁶ M⁻¹s⁻¹ for binding polypeptide associating with a ligand, thereby having improved therapeutic potency. The method can also involve the step of identifying a binding polypeptide from the one or more different progeny polypeptides having improved therapeutic potency and a kₒₙ of at least about 3×10⁶ M⁻¹s⁻¹, 5×10⁶ M⁻¹s⁻¹, 7×10⁶ M⁻¹s⁻¹, 9×10⁶ M⁻¹s⁻¹, 1×10⁷ M⁻¹s⁻¹, 3×10⁷ M⁻¹s⁻¹, 5×10⁷ M⁻¹s⁻¹, 7×10⁷ M⁻¹s⁻¹ or 9×10⁷ M⁻¹s⁻¹ or higher for binding polypeptide associating with a ligand.

A binding polypeptide that associates with a ligand and that is produced from a parent polypeptide having no measurable association rate with the ligand has an improved association rate. Specifically, a binding polypeptide improved by the methods of the invention having a measurable value for an association rate or kₒₙ that is at least 4-fold greater than the limits of detection available to the art constitutes at least a 4-fold increase in association rate or kₒₙ for the ligand. Thus, a binding polypeptide that associates with a ligand and that is produced from a parent polypeptide having no measurable association rate with the ligand is understood to have improved therapeutic potency.

The efficacy of a binding polypeptide having improved therapeutic potency can be observed in an individual to be treated or, as an alternative, in an in vivo model system including, for example, a cell based assay, a tissue based assay, or a whole organism assay. One skilled in the art will know how to determine efficacy in a model according to the conditions specific to the assay and disease under study. For example, the chick chorioallantoic membrane (CAM) assay measures angiogenesis and is a well recognized model for in vivo angiogenesis. The assay has been described in detail and has been used to measure neovascularization as well as the neovascularization of tumor tissue (Ausprunk et al., Am. J. Pathol., 79:597-618 (1975); Ossonski et al. Cancer Res., 40:2300-2309 (1980); Brooks et al. Science, 264:569-571 (1994a) and Brooks et al. Cell, 79:1157-1164 (1994b) which are incorporated herein by reference.

A binding polypeptide identified, determined or produced by the methods of the invention and having improved therapeutic potency can have improved efficacy. For example, a binding polypeptide having improved therapeutic potency as identified or determined relative to a therapeutic control of known efficacy will show improved efficacy. The methods of the invention can also be used to identify a binding polypeptide having improved therapeutic potency relative to a therapeutic control for which efficacy has not been determined as described previously. Efficacy of a binding polypeptide having improved therapeutic potency relative to a therapeutic control of unknown efficacy can be tested in an in vivo model as described above. In cases where the efficacy is less than desired the binding polypeptide can be further improved by the methods of the invention and re tested in the in vivo model. Repetition of the methods of the invention and testing in an in vivo model can be used to iteratively improve therapeutic potency of a binding polypeptide until a binding polypeptide yielding the desired efficacy is produced.

One skilled in the art will recognize that the methods of the invention, which have been exemplified herein with respect to a binding polypeptide, can be performed with any binding molecule. In this regard, one skilled in the art will know that a ligand is a binding molecule. Accordingly, a binding molecule can be identified and produced according to methods described herein with respect to identifying and producing a ligand. Thus the invention provides a method of determining the therapeutic potency of a binding molecule. The method can consist of (a) contacting a binding molecule with a ligand; (b) measuring association rate for binding between the binding molecule and the ligand, and (c) comparing the association rate for the binding molecule to an association rate for a therapeutic control, the relative association rate for the binding molecule compared to the association rate for the therapeutic control indicating that the binding molecule will exhibit a difference in therapeutic potency correlative with the difference between the association rates. The method can further consist of (d) changing one or more moiety in the binding molecule and repeating steps (a) through (c) one or more times. One skilled in the art will know that methods of combinatorial chemistry can be used in the methods of the invention to produce or change any binding molecule.

The invention further provides a method of preventing or treating a virus related disease. The method can include administering to a patient at risk thereof, or afflicted therewith, a therapeutically effective amount of an antibody or active fragment thereof of the invention.

The invention further provides a process for producing a high potency neutralizing antibody. The process includes the steps of (a) producing a recombinant antibody, including immunologically active fragments thereof, having heavy and light chain variable regions containing one or more framework and/or CDR having preselected amino acid sequences; (b) screening the recombinant antibodies for high kₒₙ when the antibody reacts in vitro with a selected antigen; and (c) selecting antibodies with the high kₒₙ. The Kₐ or kₒₙ of the antibody can be any of the values described above.

The invention further provides a method of increasing the potency of an antibody or functional fragment thereof by selectively changing one or more amino acids within the variable region framework and/or CDR regions so as to increase the measured Kₐ or kₒₙ values. Amino acid changes can be restricted to either the variable region framework or CDR regions. The Kₐ or kₒₙ of the antibody prior to or after changing amino acids can be any of the values described above and can be increased to at least the above-described values.

### EXAMPLE I

Synthesis of focused libraries of butyrylcholinesterase variants by codon-based mutagenesis.

This example describes the design and synthesis of butyrylcholinesterase variant libraries.

A variety of information can be used to focus the synthesis of the initial libraries of butyrylcholinesterase variants to discreet regions. For example, butyrylcholinesterase and Torpedo acetylcholinesterase (AChE) share a high degree of homology (53% identity). Furthermore, residues 4 to 534 of Torpedo AChE can be aligned with residues 2 to 532 of butyrylcholinesterase without deletions or insertions. The catalytic triad residues (butyrylcholinesterase residues Ser198, Glu325, and His438) and the intrachain disulfides are all in the same positions. Due to the high degree of similarity between these proteins, a refined 2.8 Å x-ray structure of Torpedo AChE (Sussman et al., Science 253: 872-879 (1991)) has been used to model butyrylcholinesterase structure (Harel et al., Proc. Nat. Acad. Sci. USA 89: 10827-10831 (1992)).

Studies with cholinesterases have revealed that the catalytic triad and other residues involved in ligand binding are positioned within a deep, narrow, active-site gorge rich in hydrophobic residues (reviewed in Soreq et al., Trends Biochem. Sci. 17:353-358 (1992)). The sites of seven focused libraries of butyrylcholinesterase variants were selected to include amino acids determined to be lining the active site gorge.

In addition to the structural modeling of butyrylcholinesterase, butyrylcholinesterase biochemical data was integrated into the library design process. For example, characterization of naturally occurring butyrylcholinesterases with altered cocaine hydrolysis activity and site-directed mutagenesis studies provide information regarding amino acid positions and segments important for cocaine hydrolysis activity (reviewed in Schwartz et al., Pharmac. Ther. 67: 283-322(1995)). Moreover, comparison of sequence and cocaine hydrolysis data of butyrylcholinesterases from different species can also provide information regarding regions important for cocaine hydrolysis activity of the molecule based on comparison of the cocaine hydrolysis activities of these butyrylcholinesterases. The A328Y mutant is present in the library and serves as a control to demonstrate the quality of the library synthesis and expression in mammalian cells.

The seven regions of butyrylcholinesterase selected for focused library synthesis (summarized in Table 2) span residues that include the 8 hydrophobic active site gorge residues as well as two of the catalytic triad residues. The integrity of intrachain disulfide bonds, located between ⁶⁵Cys-⁹²Cys, ²⁵²Cys⁻²⁶³Cys, and ⁴⁰⁰Cys⁻⁵¹⁹Cys is maintained to ensure functional butyrylcholinesterase structure. In addition, putative glycosylation sites (N-X-S/T) located at residues 17, 57, 106, 241, 256, 341, 455, 481, 485, and 486 also are avoided in the library syntheses. In total, the seven focused libraries span 79 residues, representing approximately 14% of the butyrylcholinesterase linear sequence, and result in the expression of about 1500 distinct butyrylcholinesterase variants.

TABLE 2
Summary of Butyrylcholinesterase Libraries

| Region | Location | Length | # Variants | Species Diversity |
|---|---|---|---|---|
| 1 | 68-82 | 15 | 285 | 3 |
| 2 | 110-121 | 12 | 228 | 3 |
| 3 | 194-201 | 8 | 152 | 1 |
| 4 | 224-234 | 11 | 209 | 2 |
| 5 | 277-289 | 13 | 247 | 8 |
| 6 | 327-332 | 6 | 114 | 0 |
| 7 | 429-442 | 14 | 266 | 0 |
| Total | | 79 | 1,501 | |
| | | 13.8% | | |

Libraries of nucleic acids corresponding to the seven regions of human butyrylcholinesterase to be mutated are synthesized by codon-based mutagenesis, as described above. Briefly, multiple DNA synthesis columns are used for synthesizing the oligonucleotides by β-cyanoethyl phosphoramidite chemistry, as described previously by Glaser et al., supra, 1992. In the first step, trinucleotides encoding for the amino acids of butyrylcholinesterase are synthesized on one column while a second column is used to synthesize the trinucleotide NN(G/T), where N is a mixture of dA, dG, dC, and dT cyanoethyl phosphoramadites. Using the trinucleotide NN(G/T) results in thorough mutagenesis with minimal degeneracy, accomplished through the systematic expression of all twenty amino acids at every position.

Following the synthesis of the first codon, resins from the two columns are mixed together, divided, and replaced in four columns. By adding additional synthesis columns for each codon and mixing the column resins, pools of degenerate oligonucleotides will be segregated based on the extent of mutagenesis. The resin mixing aspect of codon-based mutagenesis makes the process rapid and cost-effective because it eliminates the need to synthesize multiple oligonucleotides. In the present study, the pool of oligonucleotides encoding single amino acid mutations are used to synthesize focused butyrylcholinesterase libraries.

The oligonucleotides encoding the butyrylcholinesterase variants containing a single amino acid mutation is cloned into the doublelox targeting vector using oligonucleotide-directed mutagenesis (Kunkel, supra, 1985). To improve the mutagenesis efficiency and diminish the number of clones expressing wild-type butyrylcholinesterase, the libraries are synthesized in a two-step process. In the first step, the butyrylcholinesterase DNA sequence corresponding to each library site is deleted by hybridization mutagenesis. In the second step, uracil-containing single-stranded DNA for each deletion mutant, one deletion mutant corresponding to each library, is isolated and used as template for synthesis of the libraries by oligonucleotide-directed mutagenesis. This approach has been used routinely for the synthesis of antibody libraries and results in more uniform mutagenesis by removing annealing biases that potentially arise from the differing DNA sequence of the mutagenic oligonucleotides. In addition, the two-step process decreases the frequency of wild-type sequences relative to the variants in the libraries, and consequently makes library screening more efficient by eliminating repetitious screening of clones encoding wild-type butyrylcholinesterase.

The quality of the libraries and the efficiency of mutagenesis is characterized by obtaining DNA sequence from approximately 20 randomly selected clones from each library. The DNA sequences demonstrate that mutagenesis occurs at multiple positions within each library and that multiple amino acids were expressed at each position. Furthermore, DNA sequence of randomly selected clones demonstrates that the libraries contain diverse clones and are not dominated by a few clones.

Optimization of Transfection Parameters for Site-Specific Integration

Optimization of transfection parameters for Cre-mediated site-specific integration was achieved utilizing Bleomycin Resistance Protein (BRP) DNA as a model system.

Cre recombinase is a well-characterized 38-kDa DNA recombinase (Abremski et al., Cell 32:1301-1311 (1983)) that is both necessary and sufficient for sequence-specific recombination in bacteriophage P1. Recombination occurs between two 34-base pair loxP sequences each consisting of two inverted 13-base pair recombinase recognition sequences that surround a core region (Sternberg and Hamilton, J. Mol. Biol. 150:467-486 (1981a); Sternberg and Hamilton, J. Mol. Biol., 150:487-507 (1981b)). DNA cleavage and strand exchange occurs on the top or bottom strand at the edges of the core region. Cre recombinase also catalyzes site-specific recombination in eukaryotes, including both yeast (Sauer, Mol. Cell. Biol. 7:2087-2096 (1987)) and mammalian cells (Sauer and Henderson, Proc. Natl. Acad. Sci. USA, 85:5166-5170 (1988); Fukushige and Sauer, Proc. Natl. Acad. Sci. U.S.A. 89:7905-7909 (1992); Bethke and Sauer, Nuc. Acids Res., 25:2828-2834 (1997)).

Calcium phosphate transfection of 13-1 cells was previously demonstrated to result in targeted integration in 1% of the viable cells plated (Bethke and Sauer, Nuc. Acids Res., 25:2828-2834 (1997)). Therefore, initial studies were conducted using calcium phosphate to transfect 13-1 cells with 4 μg pBS185 and 10, 20, 30, or 40 μg of pBS397-fl(+)/BRP. The total level of DNA per transfection was held constant using unrelated pBluescript II KS DNA (Stratagene; La Jolla, Calif.), and transformants were selected 48 hours later by replating in media containing 400 μg/ml geneticin. Colonies were counted 10 days later to determine the efficiency of targeted integration. Optimal targeted integration was typically observed using 30 μg of targeting vector and 4 μg of Cre recombinase vector pBS185, consistent with the 20 μg targeting vector and 5 μg of pBS185 previously reported (Bethke and Sauer, Nuc. Acids Res., 25:2828-2834 (1997)). The frequency of targeted integration observed was generally less than 1%. Despite the sensitivity of the calcium phosphate methodology to the amount of DNA used and the buffer pH, targeted integration efficiencies observed were sufficient to express the protein libraries.

As shown in Table 3, several cell lines as well as other transfection methods were also characterized. In general, lipid-mediated transfection methods are more efficient than methods that alter the chemical environment, such as calcium phosphate and DEAE-dextran transfection. In addition, lipid-mediated transfections are less affected by contaminants in the DNA preparations, salt concentration, and pH and thus generally provide more reproducible results (Felgner et al., Proc. Natl. Acad. Sci. USA, 84:7413-7417 (1987)). Consequently, a formulation of the neutral lipid dioleoyl phosphatidylethanolamine and a cationic lipid, termed GenePORTER transfection reagent (Gene Therapy Systems; San Diego, Calif.), was evaluated as an alternative transfection approach. Briefly, endotoxin-free DNA was prepared for both the targeting vector pBS397-fl(+)/BRP and the Cre recombinase vector pBS185 using the EndoFree Plasmid Maxi kit (QIAGEN; Valencia, Calif.). Next, 5 μg pBS185 and varying amounts of pBS397-fl(+)/BRP were diluted in serum-free medium and mixed with the GenePORTER transfection reagent. The DNA/lipid mixture was then added to a 60-70% confluent monolayer of 13-l cells consisting of approximately 5×10⁵ cells/100-mm dish and incubated at 37° C. Five hours later, fetal calf serum was added to 10%, and the next day the transfection media was removed and replaced with fresh media.

Transfection of the cells with variable quantities of the targeting vector yielded targeted integration efficiencies ranging from 0.1% to 1.0%, with the optimal targeted integration efficiency observed using 5 μg each of the targeting vector and the Cre recombinase vector. Lipid-based transfection of the 13-1 host cells under the optimized conditions resulted in 0.5% targeted integration efficiency being consistently observed. A 0.5% targeted integration is slightly less than the previously reported 1.0% efficiency (Bethke and Sauer, Nuc. Acids Res., 25:2828-2834 (1997)), and is sufficient to express large protein libraries and allows expressing libraries of protein variants in mammalian cells.

TABLE 3
Expression of a single butyrylcholinesterase variant
per cell using either stable or transient cell transfection.

| Cell | | Integration | Integration? | Integration? |
|---|---|---|---|---|
| Line | Expression | Method | (PCR) | (Activity) |
| NIH3T3 | Transient | N/A | N/A | Transient, |
| (13-1) | (lipid-based) | | | very low |
| | | | | activity |
| NIH3T3 | Stable | Cre | Yes | No measurable |
| (13-1) | | recombinase | | activity |
| CHO | Transient | N/A | N/A | Transient, |
| | (lipid-based) | | | measurable |
| | | | | activity |
| | | | | (colorimetric |
| | | | | and cocaine |
| | | | | hydrolysis) |
| 293 | Transient | N/A | N/A | Transient, |
| | (lipid-based) | | | measurable |
| | | | | activity |
| | | | | (colorimetric |
| | | | | and cocaine |
| | | | | hydrolysis) |
| 293 | Stable | Flp | Yes | Measurable |
| | | recombinase | | activity |
| | | | | (colorimetric |
| | | | | and cocaine |
| | | | | hydrolysis) |

These results demonstrate optimization of transfection conditions for targeted insertion in NlH3T3 13-1 cells. Conditions for a simple, lipid-based transfection method that required a small amount of DNA and generated reproducible 0.5% targeting efficiency were established.

Expression of Butyrylcholinesterase Variant Libraries in Mammalian Cells

Each of the seven libraries of butyrylcholinesterase variants are transformed into a host mammalian cell line using the doublelox targeting vector and the optimized transfection conditions described above. Following Cre-mediated transformation the host cells are plated at limiting dilutions to isolate distinct clones in a 96-well format. Cells with the butyrylcholinesterase variants integrated in the Cre/lox targeting site are selected with geneticin. Subsequently, the DNA encoding butyrylcholinesterase variants from 20-30 randomly selected clones from each library are sequenced and analyzed as described above. Briefly, total cellular DNA is isolated from about 10⁴ cells of each clone of interest using DNeasy Tissue Kits (Qiagen, Valencia, Calif.). Next, the butyrylcholinesterase gene is amplified using Pfu Turbo DNA polymerase (Stratagene; La Jolla, Calif.) and an aliquot of the PCR product is then used for sequencing the DNA encoding butyrylcholinesterase variants from randomly selected clones by the fluorescent dideoxynucleotide termination method (Perkin-Elmer, Norwalk, Conn.) using a nested oligonucleotide primer.

As described previously, the sequencing demonstrates uniform introduction of the library and the diversity of mammalian transformants resembles the diversity of the library in the doublelox targeting vector following transformation of bacteria.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A binding polypeptide, or functional fragment thereof, comprising a kₒₙ of at least about 9×10⁷ M⁻¹s⁻¹ for associating with a ligand and having therapeutic potency.

2. A grafted antibody, or functional fragment thereof, comprising a kₒₙ of at least about 1.3×10⁶ M⁻¹s⁻¹ to a ligand and having therapeutic potency.

3. A human antibody, or functional fragment thereof, comprising a kₒₙ of at least about 9×10⁷ M⁻¹s⁻¹ to a ligand and having therapeutic potency.

4. A method of determining the therapeutic potency of a binding polypeptide, comprising:
(a) contacting a binding polypeptide with a ligand;
(b) measuring association rate for binding between said binding polypeptide and said ligand, and
(c) comparing said association rate for said binding polypeptide to an association rate for a therapeutic control, the relative association rate for said binding polypeptide compared to said association rate for said therapeutic control indicating that said binding polypeptide will exhibit a difference in therapeutic potency correlative with the difference between said association rates.

5. The method of claim 4, further comprising the step of:
(d) changing one or more amino acids in said binding polypeptide and repeating steps (a) through (c) one or more times.

6. The method of claim 5, wherein said association rate for said changed binding polypeptide increases by at least 4-fold.

7. The method of claim 4, wherein said association rate for said binding polypeptide increases correlative with improved therapeutic potency.

8. The method of claim 4, wherein said association rate for said binding polypeptide is at least 4-fold higher than said association rate for said therapeutic control.

9. The method of claim 4, wherein said association rate is indicated by kₒₙ.

10. The method of claim 9, wherein said kₒₙ for said binding polypeptide is at least about 8×10⁶ M⁻¹s⁻¹.

11. The method of claim 10, wherein said therapeutic potency correlative with the difference between said kₒₙ for said binding polypeptide and said kₒₙ for said therapeutic control is independent of an effect of a difference between Kₐ for said binding polypeptide and Kₐ for said therapeutic control.

12. The method of claim 10, wherein said difference between said kₒₙ for said binding polypeptide and said kₒₙ for said therapeutic control is an increase and Kₐ for said binding polypeptide is a similar value to Kₐ for said therapeutic control.

13. The method of claim 10, wherein said difference between said kₒₙ for said binding polypeptide and said kₒₙ for said therapeutic control is an increase and Kₐ for said binding polypeptide is a lower value than Kₐ for said therapeutic control.

14. The method of claim 4, wherein said binding polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

15. The method of claim 4, wherein said therapeutic control is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

16. A method of determining therapeutic potency of a binding polypeptide, comprising:
(a) contacting two or more binding polypeptides of a population with a ligand;
(b) measuring association rates for said two or more binding polypeptides binding to said ligand;
(c) comparing said association rates for said two or more binding polypeptides binding to said ligand, and
(d) identifying a binding polypeptide exhibiting a higher association rate for binding to said ligand than one or more other binding polypeptides of the population, said higher association rate correlating with the therapeutic potency of said identified binding polypeptide.

17. The method of claim 16, wherein said higher association rate is 4-fold higher.

18. The method of claim 16, further comprising the step of:
(d) changing one or more amino acids in said identified binding polypeptide and repeating steps (a) through (c) one or more times.

19. The method of claim 16, wherein said association rate is identified by kₒₙ.

20. The method of claim 19, wherein said kₒₙ, is at least about 1.5×10⁶ M⁻¹s⁻¹.

21. The method of claim 19, wherein said high kₒₙ is larger than kₒₙ for a therapeutic control.

22. The method of claim 16, wherein said binding polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

23. A method for producing a binding polypeptide with improved therapeutic potency, comprising:
(a) changing one or more amino acids in a parent polypeptide to produce one or more different progeny polypeptides;
(b) measuring the association rate for said one or more different progeny polypeptides associating with a ligand, and
(c) identifying a binding polypeptide from said one or more progeny polypeptides having at least a 4-fold increase in association rate to a ligand compared to the parent polypeptide, said increased association rate resulting in improved therapeutic potency toward a pathological condition.

24. The method of claim 23, wherein said association rate is indicated by kₒₙ.

25. The method of claim 24, wherein said increased kₒₙ is at least about 3×10⁵ M⁻¹s⁻¹.

26. The method of claim 24, wherein said increase in kₒₙ resulting in improved therapeutic potency is independent of an effect of a change in Kₐ for said binding polypeptide.

27. The method of claim 24, wherein said binding polypeptide having at least a 4-fold increase in kₒₙ has a Kₐ value similar to Kₐ for said parent polypeptide.

28. The method of claim 24, wherein said binding polypeptide having at least a 4-fold increase in kₒₙ has a Kₐ value lower than Kₐ for said parent polypeptide.

29. The method of claim 23, wherein said binding polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

30. The method of claim 23, wherein said parent polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

31. A method for producing a binding polypeptides with improved therapeutic potency, comprising:
(a) changing one or more amino acids in a parent polypeptide to produce one or more different progeny polypeptides;
(b) measuring the association rate for said one or more different progeny polypeptides associating with a ligand, and
(c) identifying a binding polypeptide from said one or more different progeny polypeptides having a kₒₙ of at least about 1.5×10⁶ M⁻¹s⁻¹ for binding polypeptide associating with a ligand, said binding polypeptide having improved therapeutic potency.

32. The method of claim 31, wherein said kₒₙ is at least about 9×10⁷ M⁻¹s⁻¹.

33. The method of claim 31, wherein said binding polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

34. The method of claim 31, wherein said parent polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.

35. A method of treating a pathological condition, comprising administering an effective amount of a binding polypeptide comprising a kₒₙ of at least about 9×10⁷ M⁻¹s⁻¹ for associating with a ligand.

36. The method of claim 35, wherein said binding polypeptide is selected from the group consisting of a receptor, enzyme, hormone, immunoglobulin, antibody, humanized antibody, human antibody, T-cell receptor, integrin, hormone receptor, lectin, membrane receptor, transmitter receptor, protease, oxidoreductase, kinase, phosphatase, DNA modifying enzyme, transcription factor, GTPase, ATPase, membrane channel, growth factor, insulin, cytokine, neural peptide, extracellular matrix protein and clotting factor, or functional fragments thereof.
